(19) United States
(12) Patent Application Publication
(10) Pub. No.: US 2007/0190006 A1
(43) Pub. Date: Aug. 16, 2007

(54) Methods for measuring moisture as a predictor of scalp health

(76) Inventors: Faiz Feisal Sherman, West Chester, OH (US); Vladimir Gartstein, Cincinnati, OH (US); David Burton Moore, Hamilton, OH (US); Carl Hinz Margraf III, Frankfurt am Main (DE); Brian Keith Fisher, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/514,646

(22) Filed: Sep. 1, 2006

Related U.S. Application Data
(60) Provisional application No. 60/714,166, filed on Sep. 2, 2005.

(51) Int. Cl. A61K 8/31; A61B 5/05

(52) U.S. Cl. 424/70.1; 600/417

(57) ABSTRACT

Methods provide for measuring moisture content of skin, such as the scalp. More particularly, the present invention is directed to a method comprising the steps of: (a) providing a means for measuring skin moisture content; (b) using the means for measuring skin moisture content to obtain at least one measured moisture content value for the skin; (c) correlating the measured moisture content value to a skin moisture value for the skin; and (d) using the skin moisture value to assess health state of the skin.

### CROSS REFERENCE TO RELATED APPLICATIONS

This application claims the benefit of U.S. Provisional application Ser. No. 60/714,166, filed on Sep. 2, 2005.

### FIELD OF THE INVENTION

The present invention relates generally to methods for measuring moisture content of skin. The present invention also relates to methods for measuring moisture content of scalp and methods of treating the scalp. Another aspect of the present invention, relates to methods for measuring dandruff conditions on the scalp of a consumer.

### BACKGROUND OF THE INVENTION

Skin is subject to insults by many extrinsic and intrinsic factors. Extrinsic factors include ultraviolet radiation (e.g., from sun exposure), environmental pollution, wind, heat or infrared radiation (IR), low humidity, harsh surfactants, abrasives, and the like. Intrinsic factors include chronological aging and other biochemical changes from within the skin. Whether extrinsic or intrinsic, these factors result in visible signs of skin aging and environmental damage, such as wrinkling and other forms of roughness (including increased pore size, flaking and skin lines), and other histological changes associated with skin aging or damage. Additionally, the water content of the stratum corneum has a profound influence on the appearance, flexibility, texture, and dryness of the skin, and also on the absorption of drugs and other molecules into and through the skin. The stratum corneum is the outermost layer of the epidermis, and comprises the surface of the skin.

Methods of treating the skin generally involve the application of at least one of a variety of appropriate treatments. Such treatments may be selected to provide or to restore certain desired physical or cosmetic characteristics to the skin or scalp. However, unless an appropriate treatment is selected, the desired physical or cosmetic characteristic may not be obtained.

In the case of treating skin, such as the scalp, treatments generally include shampoos, conditioners, colorants, styling compositions, and the like. Manufacturers of these scalp treatments may provide multiple versions of a type or brand of scalp treatment, wherein each of the multiple versions is specifically designed to target a need or demand which is characteristic of a specific consumer segment and which may be based on physical or cosmetic differences of the scalp generally found between respective consumer segments. For example, a single brand shampoo may offer a first version designed to treat flakes and a second version designed to treat dryness, both conditions associated with dandruff.

However, when a consumer is faced with the task of selecting a scalp treatment from among the multiple versions of a scalp care brand, the consumer may unknowingly select a version which is not designed to provide the characteristics desired by the consumer. In such a case, the consumer may be dissatisfied with the results of the selected version of the scalp care brand. As a result of the consumer's dissatisfaction, the consumer subsequently may refuse to select any of the versions of that same scalp care brand even though another version of that scalp care brand may provide the consumer's desired scalp and/or hair characteristics. The occurrence of such circumstances, in turn, may lead to unnecessary loss of sales of the particular scalp care brand for the manufacturer.

Methods of measuring moisture content have been developed in the past to determine the moisture level of skin or hair, and have relied on various techniques including electrical resistance and capacitance measurements to obtain the desired indication.

### SUMMARY OF THE INVENTION

In one embodiment, the present invention is directed to a method for measuring moisture content of skin. The method comprises the steps of: (a) providing a means for measuring skin moisture content; (b) using the means for measuring skin moisture content to obtain at least one measured moisture content value for the skin; (c) correlating the measured moisture content value to a skin moisture value for the skin; and (d) using the skin moisture value to assess health state of the skin.

In another embodiment, the present invention is directed to a method for measuring the scalp health of a consumer. The method comprises the steps of: (a) providing a device for measuring scalp moisture content; (b) using the device to obtain at least one measured moisture content value for the scalp; correlating the measured moisture content value to a scalp moisture value for the scalp; and (d) using the scalp moisture value to assess health state of the scalp.

In yet another embodiment, the present invention is directed to a method for measuring dandruff conditions on scalp. The method comprises the steps of: (a) providing a means for measuring scalp moisture content; (b) a moisture meter for measuring scalp moisture content; (b) using the means for measuring scalp moisture content to obtain at least one measured moisture content value from the head of a consumer; (c) correlating the measured moisture content value to a dandruff condition indicator value for the consumer; and (d) using the dandruff condition indicator value to assess health state of the scalp of the consumer. These and other features, aspects and advantages of the present invention will become evident to those skilled in the art from a reading of the present disclosure.

### BRIEF DESCRIPTION OF THE DRAWINGS

While the specification concludes with claims which particularly point out and distinctly claim the invention, it is believed the present invention will be better understood from the following description taken in conjunction with the accompanying drawing.

FIG. 1 is a flow diagram of the process of an embodiment of the present invention.

### DETAILED DESCRIPTION OF THE INVENTION

As used herein, the term “skin” refers to the membranous tissue forming the external covering of a mammalian body including, for example, the external covering of the face, neck, chest, back, arms, hands, legs and scalp.

As used herein, “moisture content” refers to the water present in the skin.

According to the present invention, methods are described for measuring the moisture content of skin and methods of treating skin. The determination of moisture content in skin, including scalp, is used to quantify various physical and cosmetic characteristics of the skin. For example, a scalp with a low moisture content is unhealthy and may exhibit signs of dandruff, including flakes, dryness, tightness, itchiness and/or redness/irritation. Skin hydration is a function of its normal biological activity that results in continuous moisture flux from within the body to the environment. The improvement of skin barrier function results in greater skin hydration and less moisture loss. As a result, the physical and cosmetic characteristics of skin may be improved, for example, with treatments that restore skin to normal conditions and improve its barrier function. Improvement of barrier function, in turn, also results in protecting the skin from environmental, physical, chemical, or biological insults and results in an overall improvement in skin health.

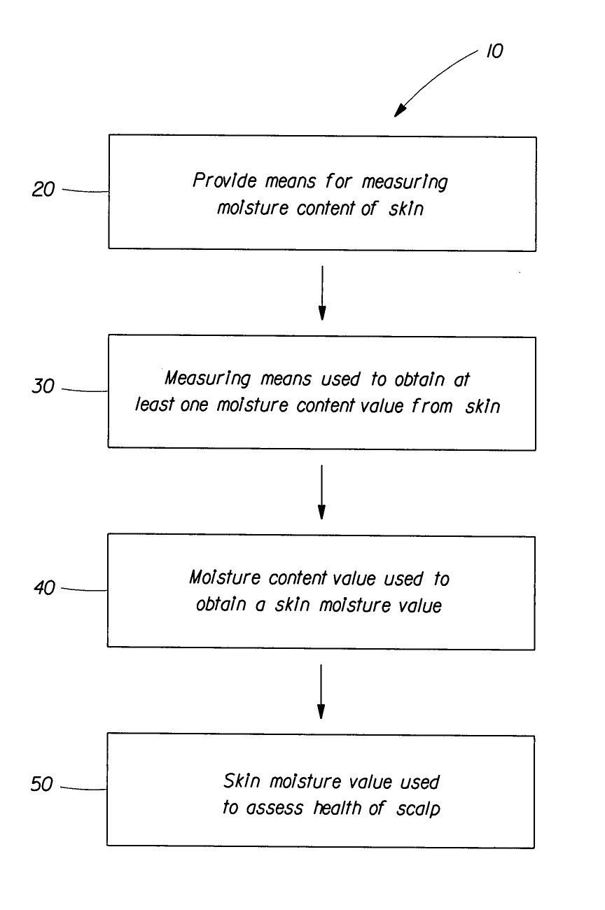

Referring now to FIG. 1, method for measuring moisture content of skin 10, contains a series of steps which are intended to provide information to a recipient or user for purposes of assessing the moisture level of skin. In a first step 20, a means for measuring skin moisture content is provided. Any suitable means for measuring skin moisture content may be used. For example, the means for measuring skin moisture content may include radio frequency, infrared, nuclear magnetic resonance, mechanical vibration, skin deformation, iontophoresis, topology, friction, trans epidermal water loss (TEWL), optical and heat dissipation devices. Various means for measuring skin moisture that may be used in accordance with the present invention are described in more detail below:

Radio Frequency (RF)

Electromagnetic fields generated by RF sources have a very strong interaction with water molecules. Very little moisture can have large effects on the permittivity of materials. This permittivity can be measured using RF fields. The higher the moisture level, the higher the permittivity of the material.

Infrared (IR)

Infrared refers to an area in the electromagnetic spectrum extending beyond red light. In one embodiment, a device delivers light at wavelengths that either absorb moisture, for example, 1200 nm, 1450 nm and 1940 nm) or does not absorb moisture, for example, reference wavelengths (1300 nm). The wavelengths are then reflected off the skin and measured by a detector. The ratio of absorbed to reference light is proportional to moisture content.

Nuclear Magnetic Resonance (NMR)

It is a phenomenon exhibited when nuclei possess spin and therefore a magnetic moment. This phenomenon in nuclei such as hydrogen protons causes them to absorb energy from a RF source at certain characteristic frequencies, when placed in a external magnetic field. The position of the nuclei in the molecule effects the electronic environment of the nucleus and thus effects the absorption frequency. The frequency differences observed in the resultant spectrum define the molecular structure of the sample, for example the water content of skin.

Mechanical Vibration

In one embodiment, a device applies a mechanical wave (wide range of frequency including sonic) to the skin and then senses the reflected wave back. The wave propagation is a function of the mechanical property of skin. It is known in the art that the skin's young's modulus changes as a function of hydration. The more hydrated the skin the lower the value and greater the dissipation of a wave, therefore a lower reflected value on more hydrated skin.

Skin Deformation

In one embodiment, a device measures the deformation of skin. It is known in the art that the skin's young's modulus changes as a function of hydration. The more hydrated the skin the lower the value. By displacing the skin and monitoring the reactive force, hydration or moisture level can be determined.

Iontophoresis

Iontophoresis is the introduction of ions into the human tissues, or exchange of ions within the tissues, by means of an electric current. The skin can be thought of as having an element equivalent to a electronic resistor. The resistance value of the skin is strongly dependent on its hydration level. The more moisture in the skin the less the resistance. By means of iontophoresis, one skilled in the art can monitor the value of current necessary to pass through the skin (resistor). This current level can be used to then determine the hydration or moisture level of skin.

Optical

In one embodiment a magnified image of the skin, for example 50×, is taken and compared to an image of healthy skin. Dry skin appears diffusive with up-lifted scales having an overall flat texture, while healthy skin appears glossy with a very well defined texture.

Friction

As skin gets dry its texture disappears giving it a flat topology. In general, this increases the surface friction. The opposite is true for healthy skin. As a result, a friction sensing device can discriminate dry vs. healthy skin by measuring the surface friction.

Trans Epidermal Water Loss

Trans Epidermal Water Loss (TEWL) is a measure of the amount water being evaporated from the skin. In one embodiment, a device, for example, a VapoMeter, may measure and provide TEWL values.

Surface Topology

In one embodiment, surface topology can be measured by friction, optical, capacitive based sensors and/or a combination of two or more of the previously mentioned methods.

Heat Dissipation

In one embodiment, a device applies a heat pulse to the skin and then senses temperature dissipation as a function of time. Water is a great heat sink. The greater the water content, the quicker the dissipation. By examining the dissipation profile the level of skin hydration can be determined.

In a second step 30, the means for measuring skin moisture content is used to obtain at least one measured moisture content value for the skin. In another example, more than one measured moisture content value is obtained for the skin. As each measurement is captured by the measurement means it is displayed or stored in a suitable manner. In one example, the measured moisture content values are displayed on an LED display. In another example, the measured moisture content values are stored in a programmable integrated circuit.

In a third step 40, the moisture content value is correlated to a skin moisture value. In one embodiment, the measurement means is electrically coupled through a cable to a processing system, such as a conventional PC or laptop computer. In another embodiment, the measurement means is electrically coupled to a programmable integrated circuit. In yet another embodiment, the measurement means is electrically coupled to a computer wirelessly. In one example, the processing system is operable to convert a measurement value, such as frequency, into a skin moisture value as shown in Table I.

TABLE I

| Measured Signal | Skin Moisture | Skin |
|---|---|---|
| Value (MHz) | Value | Condition |
| >=0.9 | 5 | D |
| 0.79-0.89 | 10 | D |
| 0.68-0.78 | 20 | D |
| 0.54-0.67 | 30 | D |
| 0.43-0.53 | 40 | D |
| 0.34-0.42 | 50 | M |
| 0.25-0.33 | 60 | M |
| 0.15-0.24 | 70 | M |
| 0.06-0.14 | 80 | M |
| 0.04-0.05 | 90 | H |
| <=0.03 | 95 | H |

In a fourth step 50, the skin moisture value is used to assess the health of the skin. In one embodiment, the skin moisture value is from about 0 to about 99, and in another embodiment, from about 5 to about 95. The skin moisture value may also be represented by such terms as “healthy” or “H”, “moderate” or “M”, and “dry” or “D,” however, any words or a numbered grade scale which depict increasing or decreasing quantities of skin moisture levels may be used in the invention.

Example of Method Using RF Based Device

An example of a method of the present invention using a RF device is described below. The measurement of the moisture content of the skin is made for at least two areas on the skin, a control area that is in equilibrium with the environment and a test area. In one example, the control skin area may include the face, neck, ears, arms, hands, legs, feet, abdomen, back and groin, while the test area may include, for example, the face, neck, chest, back, arms, hands, feet, legs and scalp. However, several such measurements at various positions on the test area can be produced. In one example, the device is used to obtain one or more additional measured moisture content values on the test area. In one embodiment, the measured values obtained for the control area and the test area have a frequency of less than about 5 MHz, in another embodiment, less than about 2.4 MHz, and in yet another embodiment, less than about 0.5 MHz.

The at least two measured moisture content values are compared to each other in order to obtain a measured moisture content differential. For example, the skin moisture content value for the control area (m1) is compared to the skin moisture content value for the test area (m2), to obtain a measured moisture content differential (m2−m1) or (m1−m2). In one embodiment, the measurement device is electrically coupled through a cable to a processing system, such as a conventional PC or laptop computer. In another embodiment, the measurement device is electrically coupled to a programmable integrated circuit. In yet another embodiment, the measurement device is electrically coupled to a computer wirelessly. The processing system is operable to convert the measured moisture content values generated by the measurement device into a measured moisture content differential. In another embodiment, the measured moisture content differential may be determined manually using the formula (m2−m1) or (m1−m2).

If additional test area skin moisture values are obtained, all of these values are compared to one another and the measurement with the largest value, lowest value or mean value is then compared with the skin moisture content value for the control area in order to obtain a measured moisture content differential profile. In one embodiment, a measurement differential from about 0 MHz to about 0.05 MHz, represents a healthy skin condition (high moisture level); a measurement differential from about 0.05 MHz to about 0.5 MHz represents a moderate skin condition (medium moisture level); and a measurement differential greater than about 0.5 MHz represents an unhealthy skin condition (low moisture level). In another embodiment, the control area and the test area could be the same. For example, a measurement of the moisture content is taken at the control area. A period of time is then allowed to elapse during which the control area may be treated with a skin or scalp treatment. Thereafter, a second measurement is taken at the control area. The two measured moisture content values are then compared to each other in order to obtain a measured moisture content differential. In effect, the control area and the test area are the same for the second measurement.

In another aspect, the invention relates, additionally, to using the skin moisture value to select at least one appropriate skin treatment. As used herein, an “appropriate skin treatment” is a chemical composition or non-chemical treatment which provides or restores skin with the physical or cosmetic characteristics desired by a consumer. Exemplary skin treatment chemical compositions may include, without limitation, beauty care products, healthcare products, cosmetics, baby care products, feminine care products and pet care products, in the form of, lotions, creams, gels, tonics, after shave, sticks, sprays, ointments, pastes, powders, mousse, shampoos, conditioners, oils, colorants, and biomedical and dermatological treatments.

Nonlimiting examples of biomedical and dermatological treatments include prescription skin care treatments, laser treatment, chemical peel, dermabrasion, electrical stimulation, botox treatments, surgical treatments and exfoliating pads and cloths.

Exemplary non-chemical treatment may include, without limitation, hair shaving and hair removal.

In another embodiment, a method for measuring the scalp health of a consumer contains a series of steps which are intended to provide information to a recipient or user for purposes of assessing the moisture level of the scalp. In a first step, a device, for example, a radio frequency device, for measuring scalp moisture content is provided. In one embodiment, the measured value obtained from the skin has a frequency from about 0 MHz to 5 Mhz, in another embodiment, from about 1 MHz to about 3 MHz, and in yet another embodiment, from about 1.5 MHz to about 2.5 MHz.

In a second step, the device is used to obtain at least one measured moisture content value for the scalp. In a third step, the moisture content value is correlated to a scalp moisture value for the scalp, in the same manner as described above with respect to the skin moisture value. In a fourth step, the scalp moisture value is used to assess the health state of the scalp. In one embodiment, the scalp moisture value is from about 0 to about 99, and in another embodiment, from about 5 to about 95. The scalp moisture value may also be represented by such terms as “healthy” or “H”, “moderate” or “M”, and “dry” or “D,” however, any words or a numbered grade scale which depict increasing or decreasing quantities of scalp moisture levels may be used in the invention.

In another aspect of the invention, the method of this invention relates to a method of treating the scalp, including (a) measuring moisture content of said scalp according to the method described above; (b) using the scalp moisture value to select at least one appropriate scalp treatment; and (c) applying the appropriate scalp treatment to the scalp. As used herein, an “appropriate scalp treatment” is a treatment which provides or restores the scalp with the physical or cosmetic characteristics desired by a consumer. Exemplary scalp treatment compositions may include, without limitation, shampoos, conditioners, oils, colorants, and styling compositions.

In another embodiment of the invention, the method of this invention relates to a method for measuring dandruff conditions on the scalp, including (a) providing a means for measuring scalp moisture content; (b) using said means to obtain at least one measured moisture content value from the head of a consumer; (c) correlating the measured moisture content value to a dandruff condition indicator value; and (d) using the dandruff condition indicator value to assess degree of dandruff on the scalp of the consumer. In one example, dandruff conditions may include, for example, flakes, dryness, tightness, itchiness and redness/irritation. In one embodiment, the dandruff condition indicator value is from about 0 to about 99, and in another embodiment, from about 5 to about 95. The dandruff condition indicator value may also be represented by such terms as “healthy” or “H”, “moderate” or “M”, and “dry” or “D,” however, any words or a numbered grade scale which depict increasing or decreasing quantities of scalp moisture levels may be used in the invention.

In another embodiment, biomarkers, other than the measurement of moisture content of skin, may serve as predictors of skin health and, further, scalp health. Nonlimiting examples of such biomarkers may include proteins, lipids, antibodies, micro organisms, pH, and ionic strength.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for measuring moisture content of skin, said method comprising the steps of:
a) providing a means for measuring skin moisture content;
b) using said means for measuring skin moisture content to obtain at least one measured moisture content value for said skin;
c) correlating said measured moisture content value to a skin moisture value for said skin; and
d) using said skin moisture value to assess health state of said skin.

2. The method according to claim 1, wherein said means for measuring skin moisture content is a device selected from the group consisting of radio frequency, infrared, nuclear magnetic resonance, mechanical vibration, skin deformation, iontophoresis, optical, friction, surface topology and heat dissipation.

3. The method according to claim 1, further comprising the step of using said skin moisture value to select at least one appropriate skin treatment.

4. The method according to claim 3, wherein said appropriate skin treatment is selected from the group consisting of chemical compositions and non-chemical treatments.

5. The method according to claim 4, wherein said chemical compositions are selected from the group consisting of lotions, creams, gels, tonics, after shave, sticks, sprays, ointments, pastes, powders, mousse, shampoos, conditioners, oils, colorants, and biomedical and dermatological treatments.

6. The method according to claim 4, wherein said non-chemical treatment is selected from the group consisting of hair shaving and hair removal.

7. A method for measuring scalp health of a consumer, said method comprising the steps of:
a) providing a device for measuring scalp moisture content;
b) using said device to obtain at least one measured moisture content value for said scalp;
c) correlating said measured moisture content value to a scalp moisture value for said scalp; and
d) using said scalp moisture value to assess health state of said scalp.

8. The method according to claim 7, wherein the device is a radion frequency device.

9. The method according to claim 8, wherein said moisture content value has a frequency of from about 0 MHz to about 5 MHz.

10. The method according to claim 8, wherein said moisture content value has a frequency of from about 1.5 MHz to about 2.5 MHz.

11. A method of treating the scalp, said method comprising the steps of:
a) measuring moisture content of said scalp according to the method of claim 7;
b) using said scalp moisture value to select at least one appropriate scalp treatment; and
c) applying said appropriate scalp treatment to said scalp.

12. The method according to claim 11, wherein said appropriate scalp treatment is selected from the group consisting of shampoos, conditioners, colorants, oils and styling compositions.

13. A method for measuring dandruff conditions on scalp, said method comprising the steps of:
a) providing a means for measuring scalp moisture content;
b) using said means for measuring scalp moisture content to obtain at least one measured moisture content value from head of a consumer;
c) correlating said measured moisture content value to a dandruff condition indicator value for said consumer; and
d) using said dandruff condition indicator value to assess degree of dandruff on scalp of said consumer.

14. The method according to claim 13, wherein said means for measuring scalp moisture content is a device selected from the group consisting of radio frequency, infrared, nuclear magnetic resonance, mechanical vibration, skin deformation, iontophoresis, optical, friction, surface topology and heat dissipation.

15. The method according to claim 13, further comprising the step of using said dandruff condition indicator value to select at least one appropriate scalp treatment.

16. The method according to claim 15, wherein said scalp treatment is selected from the group consisting of shampoos, conditioners, oils, colorants, and biomedical and dermatological treatments.
